(19) United States
(12) Patent Application Publication
(10) Pub. No.: US 2008/0045477 A1
(43) Pub. Date: Feb. 21, 2008

(54) Series Of New 4,6-Diamino-1,2-Dihydro-1-Aryl-1,3,5-Triazines, Substituted By An Adamantyl Moiety In The Position 2 Of Triazine-Their Corresponding Salts, Isomers,Steroisomers, Enantiomers, Free Bases And The Complexes Of All The Above With Natural Macromolecules And Their Synthetic Derivatives.

(76) Inventors: Ekaterini Antoniadou-Vyza, Athens (GR); Polyvxeni Tsitsa, Athens (GR); Christos Roussakis, Nantes (FR)

Correspondence Address: WATOV & KIPNES, P.C., P.O. BOX 247, PRINCETON JUNCTION, NJ, 08850, US

(21) Appl. No.: 10/480,868

(22) PCT Filed: Jun. 14, 2002
(86) PCT No.: PCT/GR02/00036

§ 371 Date: Aug. 17, 2007

(30) Foreign Application Priority Data
Jun. 15, 2001 (GR) 20010100290

(51) Int. Cl. A61K 31/53; A61K 47/48; A61P 35/00; C07D 251/10

(52) U.S. Cl. 514/58; 514/245; 536/103; 544/208

(57) ABSTRACT

A series of new 4,6-diamino-1,2-dihydro-1-aryl-2-(-tricyclo[3.3.1.1^{3,7}]decyl]-1,3,5-triazines where the aryl group is a substituted or unsubstituted phenyl, or naphthyl-group, as pharmaceutically accepted salts or as free bases. The phenyl group substituents are halides, alkyls, alkoxyls or nitro group. One or more of the above substituents are found one or more times each one, and in various positions of the phenyl group. The corresponding isomers, stereoisomers, enantiomers, free bases and their complexes with various natural macromolecules and synthetic derivatives of them. The invention refers to new and prototype compounds inhibiting the proliferation of unwanted cells that belong to pathogenic microorganisms, to human tissues, to pathological cells, or any pathological cell proliferation. It also refers to the synthesis and the method of synthesis of the above mentioned compounds. The synthesis and the method of synthesis thereof of a series characterized by the preparation of the final triazines, by cyclization of the bigouanide hydrochlorides with adamantane-1-carboxaldehyde. Preparation of the inclusion complexes is realized from compexation of the corresponding hydrochlorides of the above mentioned triazines with natural macromolecules or synthetic derivatives.

### The present invention refers to new and prototype molecules, of the class of 4,6-diamino symmetrical triazines, substituted by an adamantyl- in position 2 of triazine according the claims 1-3, to their synthetic preparation and production from synthesis, and to the inclusion of the above compounds in natural macromolecules, e.g. cyclodextrins or their synthetic derivatives.

These new molecules are acting as inhibitors of the cell proliferation. Their activity is probably due to inhibition of the enzyme dihydrofolate reductase, or to other biological processes as the expression of genes, inhibiting the growth of unwanted tissues and cells that belong to microorganisms as well as unwanted cells belonging to the host, human organism, tumor cells or cells of other kinds, and they exhibit antimicrobial, anti-parasitic, and anticancer activity.

Several compounds are known as inhibitors of this enzyme, acting by inhibiting the growth and the proliferation of unwanted tissues and cells belonging to different microorganisms, as well as hosts unwanted cells, belonging to the human organism, tumor cells or cells of other kinds.

The lack of specific and selective drugs creates serious problems in dealing with diseases like the encephalitis caused by Toxoplasma gondii (T.g.), the human bronchopulmonary carcinoma, or the secondary cataract.

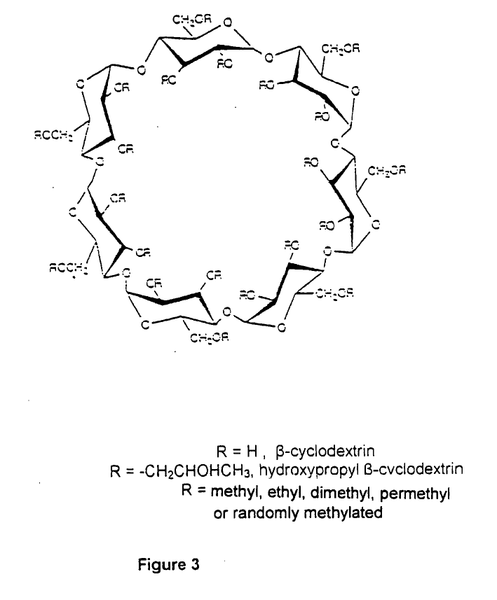

We have therefore designed molecules of the general type A, with enhanced lipophilicity both in the overall lipophilicity, and in the lipophilicity of the substituent on position 2 of triazines. The present invention consists of the synthesis and production of the aforementioned compounds of the category of the 4,6-diamino -1-aryl—symmetric triazines, of the general type A, as free bases and as pharmaceutically accepted salts, as well as of the synthesis and production of the complexes of the compounds of the general type A, with different natural cyclodextrins or with their synthetic derivatives of the type described in FIG. 3. In these synthesized molecules the basic structure remains the same, but when a phenyl group exists in the position 1 of the triazine, the substituents on the phenyl ring vary.

In the position 1 of the triazine there is a 1-naphthyl group, a 2-naphthyl group, or a phenyl group. In the latter case, the substituents are halides e.g.: chlorine, bromine, fluorine, iodine, alkyls e.g.: methyl, ethyl, etc., as well as alkoxyls e.g.: methoxyls, ethoxyls, etc., or the nitro group. One or more of the above substituents are found one or more times each one, and in various positions of the phenyl group.

In the position 2 of the triazine ring an adamantyl moiety exists.

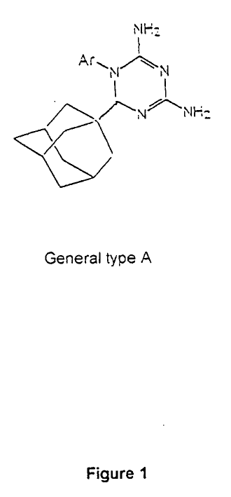

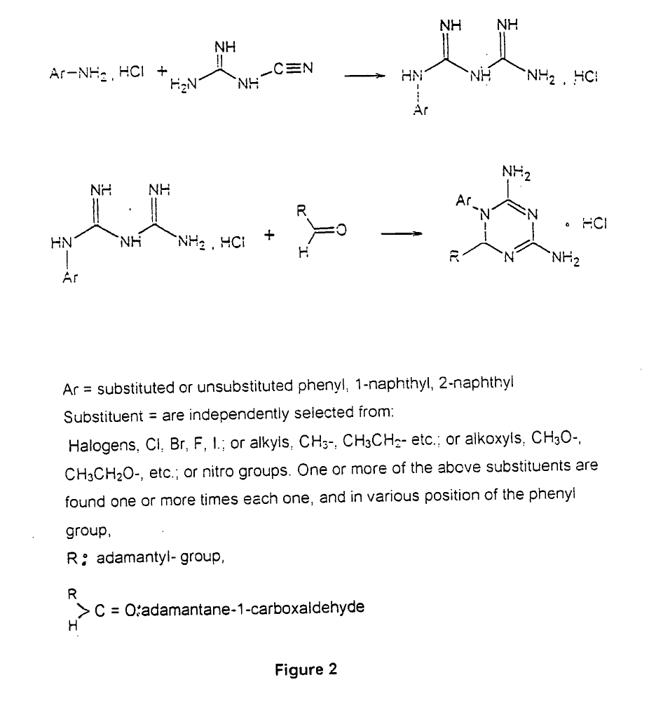

The synthetic preparation of the new triazine molecules as well as the intermediates can be achieved by following the method of organic synthesis described in FIG. 2 by the following preparation steps;

a) Preparation of the adamantane-1-methanol, by reduction of the adamantane-1-carboxylic acid in the presence of lithium aluminum hydride in tetrahydrofuran as solvent.

b) Preparation of the adamantane-1-carboxaldehyde by oxidation of the adamantane-1-methanol with pyridiniumchlorochromat in dichloromethan as a solvent.

c) Preparation of the bigouanide hydrochlorides with aromatic substituent, precursors of the final triazines, by fusion of the primary aromatic amine hydrochlorides with dicyandiamide, by continuous heating for several hours, by boiling or refluxing the corresponding compounds.

And characterized by cyclization of the bigouanide hydrochlorides with adamantane-1-carboxaldehyde, diluted in various solvents and in the presence of acid as catalyst, by heating, refluxing for several hours, or fusion of the reactants.

In some cases the preparation of final compounds, instead of using step c and the final cyclization can be also realized according to the one single step method, by mixing the three reactants followed by fusing, or refluxing in appropriate solvent.

The progress and the end point of the reaction are detected with NMR Spectroscopy.

All free bases are obtained through appropriate treatment of the corresponding hydrochlorides produced from the preparation reaction.

All the prepared triazine molecules, are converted to the corresponding inclusion complexes with natural macromolecules or their synthetic derivatives, e.g. cyclodextrins (FIG. 3).

The preparation of the complexes in the case of cyclodextrins is realized by adding an amount of the active compound to an aqueous solution of the corresponding cyclodextrins under continuous stirring. The complexation procedure is completed by continuous stirring. The time needed for completion is different for each molecule, depending on their physicochemical properties, the volume of their substituents and the lipophilicity of their included part.

Another means of improving the properties of the compounds is through complexation with the polymers, synthetic or biological.

The complexation is confirmed by phase solubility stadies as well as by NMR Spectroscopy. The observation of the chemical shift changes of the protons of both the guest and the host molecules, specially of the internal protons of cyclodextrin molecules is an evidence of complexation. By using the titration technique, i.e. adding increasing quantities of triazine in aqueous (D₂O) solution of cyclodextrin, a continuous shift was observed at the signals corresponding to H3 and H5, located inside the cavity, contrary to the H2 and H4, located at the external surface, which remain at the same frequency.

NMR Spectroscopy was also used in the determination of the complex stoichiometry, which was found to be 1:1 in all cases, either in the same place or alternating. The stoichiometry is determined using the continuous variation method, a method of assessment of the plots produced by measuring the chemical shift changes in selected protons, known as Job's plot.

### EXAMPLE

4,6-Diamino-1,2-dihydro-1-(4-chlorophenyl)-2-(1-tricyclo[3.3.1.1^{3,7}]decyl)-1,3,5-triazine hydrocloride. (of the general type A)

a) In a stirred suspension of 2.53 gr of lithium aluminum hydride in 50 ml tetrahydrofuran, cooled at 0° C., 6 gr of adamantane-1-carboxylic acid diluted in 25 ml tetrahydrofuran are added dropwise. The reaction mixture is refluxed for 24 h and then cooled in 0° C. and hydrolyzed by adding water and 20% aqueous solution of sodium hydroxide. The precipitate of inorganic salts is filtered, rinsed with tetrahydrofuran and the filtrate is condensed in vacuum. The residue is diluted in ethyl ether, washed with aqueous solution of sodium carbonate. The organic phase is dried over sodium sulfate and the solvent is removed by distillation. The obtained adamantane-1-methanol, is recrystallized from ethyl ether.

Yield=96%.

b) 3.5 gr of adamantane-1-methanol diluted in 40 ml of anhydrous dichloromethan, are added to a rigorously stirred suspension of 6.83 gr pyridiniumchlorochromate in 40 ml anhydrous dichloromethan. The reaction mixture is stirred for 90 min in ambient temperature and then 40 ml dichloromethan are added. After stirring for an additional 5 min the mixture is filtered from neutral silica. The combined organic layers are concentrated in vacuum. The residue is obtained with 30 ml of pentane which is evaporated in 25° C. in vacuum. A 93% yield of adamantane-1-carboxaldehyde is thus obtained and used in the cyclization reaction without further purification.

c) Method of preparation of the corresponding bigouanides: 1 mole of hydrochloric salt of a primary aromatic amine reacts with 1.07 moles of dicyandiamide under stirring and refluxing for several hours, using propyl alcohol as solvent. The reaction time is dependent on the amine used each time. The bigouanides can also be prepared by fusing the above mentioned reactants.

Preparation of the final compound:

A mixture of 2.48 gr (0.01 mole) of 4-chlorophenyl bigouanide hydrochloride, 0.5 ml (0.005 mole) of concentrated hydrochloric acid and 1.64 gr (0.01 mole) of adamantane-1-carboxaldehyde in 10 ml of absolute ethanol as solvent is refluxed with stirring for several hours. After the end of the reaction the mixture is condensed and the recrystallization of the precipitate from ethanol from ethanol gives pure product with m.p. 237-9° C. Yield=68%.

Molecular formula=C₁₉H₂₅N₅Cl₂. Molecular weight=382.34

4,6-Diamino-1,2-dihydro-1-(4-chlorophenyl)-2-(1-tricyclo[3.3.1.1^{3,7}]decyl)-1,3,5-triazine

The free base is obtained from the above hydrocloride by suitable treatment. Molecular formula=C₁₉H₂₄N₅Cl. Molecular weight=345.87.

m.p.=168-170° C.

Inclusion complex of 4,6-diamino-1,2-dihydro-1-(4-chlorophenyl)-2-(1-tricyclo[3.3.1.1^{3.7}]decyl)-1,3,5-triazine hydrochloride with β-cyclodextrine

Equimolecular quantities of the above triazine and the β-cyclodextrin are brought to an erlenmayer and a small quantity of distilled water is added with simultaneous heating to 30° C. after stirring for several hours, in order to achieve the completion of complexation. The mixture is left to rest until all the amount of the complex precipitates in crystalline form. The precipitate is filtered, washed with small quantities of water and diethyl ether, and then dried over phosphorous pentoxide.

Molecular formula=C₁₉H₂₅N₅Cl₂.C₄₂H₇₀O₃₅. Molecular weight of the complex=1517.34. Yield 100%

Inclusion complex of 4,6-diamino-1,2-dihydro-1-(4-chlorophenyl)-2-(1-tricyclo[3.3.1.1^{3,7}]decyl)-1,3,5-triazine hydrochloride with hydroxypropyl pβcyclodextrine.

It is prepared by the same mixture as in the case of the β-cyclodextrin complex, which is then liophilized.

Molecular formula=C₁₉H₂₅N₅Cl₂.C₆₃H₁₁₂0₄₂ Molecular weight of the complexes=1882.34. Yield 100%

### PHARMACOLOGICAL DATA

The concentration of the studied compounds required to inhibit the activity of the enzyme DHFR (from chicken) by 50%, IC_{50,} was in the range of 2×10⁻⁴ M to 9.7×10⁻⁵ M.

In the case of the microorganism Toxoplasm gondii, for the studied compounds, the concentration required to inhibit the growth by 50%, IC₅₀, was found to be from 1×10⁻⁴ M to 1×10⁻⁵ M.

In the case of cytotoxic and anticancer activity, the studied compounds at the realized experiments show that the concentration required for the growth inhibition of the various cancer cell lines was:

The (NSCLC-N6).L16 cell line, derived from a primary culture of moderately differentiated, rarely keratinized human non-small-cell bronchopulmonary carcinoma (μg/ml) 3.4, <3.3, 7.9, <3.3, 8.12, 3.66, <3.3, 8.93, >30, 13.38, 7.63, >30, 11.21.

C98 (μg/ml) 4.6, 3.8, 13.9, <3.3, >30, 11.1, 11.3, 18.3, 16.9, 13.

Cytotoxicity against human lens epithelial cell line SRA 01/04 (μg/ml) <2.7, 5.2, 26.04.

### Experimental Testing Method

The cell line utilized for the following experiments is derived from epithelial cells of human lens SRA 01/04, which was established by transfection with large T-antigen of virus SV40 (Ibaraki N. et al 1998). It is cultured in antibiotic-free Dulbecco's modified Eagle's medium (DMEM) supplemented with 4% fetal calf serum. Incubation is carried out at 37° C., in a humidified atmosphere with 5% CO₂.

### Compound Selection

Initially, a compound is selected, which ceases the SRA 01/04 cell proliferation. The compound to be tested will be dissolved in the means previously described. For this reason, four in vitro experiments took place:

The experiment, IC₅₀, allows us to detect the concentration of the tested compound required to reduce cell proliferation by 50%. The values are expressed in μg/ml.

In order to determine this concentration, the cells are exposed to the drug for 72 hours, in 96-well microtiter plates (6×10⁴ cell/ml) in three different concentrations (3, 3, 10, and 30 λg/ml). After 72 hours the cytotoxicity is measured by a calorimetric assay based on the conversion of tetrazolium dye MTT (3-4,5-dimethylthiazol-2-yl-2,5-diphenyltetrazolium bromide) to a blue-black formazan product, at 570 nm. Optical density is read on a Titertek Multiscan MKII. Compounds having IC₅₀ in the range of 3,3 and 30 μg/ml was further tested.

The experiment of growth kinetics allows us to evaluate the mode of action of the compounds in a period of time of 72 hours. The experimental procedure is as described above. The Optical Density is measured every 24 hours, in the presence of four different concentrations of each substance and the % growth is calculated.

The irreversibility experiments allow us to observe the irreversibility of the compounds activity. After the cell treatment with the active compound the cells cannot regain their proliferation. Such compounds are called, final differentiation inducers (G₁−dt). In this stage the cells cannot re-enter to the cell cycle.

The study is divided in two periods. Initially the cell is treated for 72 hours with the compounds that exhibit significant activity. The cells are then cultured for 72 hours in normal media (DMEM σε 4% SVF).

In order to understand the exact time of the cell cycle the compounds are acting, the flow cytometry assay is realized. The retained compounds are inducing the final differentiation of the cell. (G₁×dt).

We attempt to verify the exact time in which the cell division hase ceased. After 72 hours of treatment with a selected compound the cell DNA appears, with propidium iodide.

The tested compounds show significant enhancement of their activity when they are included or incorporated in macromolecules .eg. in the form of inclusion complexes of natural cyclodextrins, or in the form of inclusion complexes of cyclodextrin synthetic derivatives, such as: hydroxypropyl-, methyl-, permethyl-, dimethyl-, randomly methylated, sulfated, ionic, non ionic; or various synthetic or biological macromolecules or polymers or not.

All the experimental data presently available allow us to believe that the above mentioned molecules can have, therapeutically significant, results.

### REFERENCE FOR THE PHARMACOLOGICAL PART

Ibaraki, N., Chien, S. C., Lin, L., Okamoto, H., Pipas, J. M. and Reddy, V. (1998)—Human lens epithelial cell line. Exp. Eye. Res., 67 577-585.

Liang, P. and Pardee, A. B. (1992)—Differential display of eukariotic messager RNA by means of the polymerase chain reaction.—Science, 257: 967-71.

Marcantonio, J. M. and Vrensen, G. F. J. M. (1999)—cell biology of posterior capsular opacification.—Eye, 13:484-88.

Sanger, F., Nicklen, S., Coulson, A. R. (1977)—DNA sequencing with chain-terminating inhibitors.—Proc. Natl. Acad. Sci., 74 (12): 5463-7 Saraux, H. (ed. 6) (1995)—Abrege d'ophtalmologie—Masson, Paris. P 103-110.

Schaumberg, D. A., Reza Dana, M., Christen, W. G. and Glynn, R. J. (1998)—A systematic overview of the incidence of posterior capsule opacification. —Ophtalmology, 105 (7): 1213-21.

Spalton, D. J. (1999)—Posterior capsular opacification after cataract surgery.—Eye, 13 (Pt 3b): 489-92.

West, S. (2000)—Looking forward to 20/20: a focus on the epidemiology of eye diseases.—Epidemiol. Rev. 22 (1): 64-70.

1. A series of new 4,6-diamino-1,2-dihydro-1-aryl-2-(1- tricyclo[3.3.1.1^{3,7}]decyl]-1,3,5-triazines where the aryl group is a substituted or unsubstituted phenyl, or naphthyl group, in the form of pharmaceutically accepted salts or in the form free bases, of the general type A 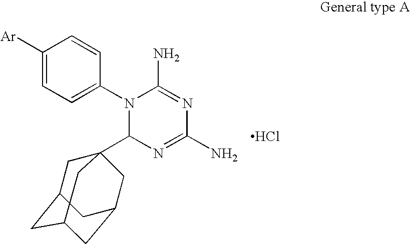
were the phenyl group substituents are halides e.g.: chlorine, bromine, fluorine, iodine, they are alkyls e.g.: methyls, ethyls, etc. as well as alkoxyls like: methoxyls, ethoxyls, etc., or the nitro group; one or more of the above substituents are found one or more times each one, and in various positions of the phenyl group.

2. Pharmaceutically accepted salt or free base of 4,6-diamino-1,2-dihydro-1-(4-chloro-phenyl)-2-(1-tricyclo[3.3.1.1^{3,7}]decyl]-1,3,5-triazine.

3. Compounds according to claims 1 and 2, either in the form of an inclusion complex with natural cyclodextrins alpha, beta, gamma, etc. or with synthetic derivatives of the natural cyclodextrins, such as hydroxypropyl-, methyl-, sulfated, ionic or non ionic derivatives, etc., and in all possible stoichiometries, incorporated in synthetic or biological macromolecules or polymers, or not.

4. The synthesis and the method of synthesis thereof of a series of new and prototype molecules, the 4,6-diamino-1,2-dihydro-1-aryl symmetrical triazines, substituted by the adamantyl moiety in the position 2 of the triazine ring, according to claims, 1, 2 and 3; the method of synthesis includes the preparation steps a, b and c and characterized by the cyclization reaction of bigouanides with the adamantane-1-carboxaldehyde:
a) preparation of the adamantane-1-methanol, by reduction of the adamantane-1-carboxylic acid in the presence of lithium aluminum hydride as a catalyst, in tetrahydrofuran as solvent; b) preparation of the adamantane-1-carboxaldehyde by oxidation of the adamantane-1-methanol with pyridiniumchlorochromat in dichloromethan as a solvent; c) preparation of the bigouanide hydrochlorides, precursors of the final triazines, by fusion of the primary aromatic amine hydrochlorides with dicyandiamide, by continuous heating for several hours, or by boiling the corresponding compounds;
and it is characterized by the cyclization of the bigouanide hydrochlorides with adamantane-1-carboxaldehyde, diluted in various solvents and in the presence of acid as catalyst, by heating, refluxing for several hours, or fusion of the reactants.

5. The synthesis and the method of synthesis thereof of 4,6-diamino-1,2-dihydro-1-aryl-2-(1- tricyclo[3.3.1.1^{3,7}]decyl]-1,3,5-triazines, of the claims 1, 2 and 3, according to the previous claim which characterized by one single step, either by fusing the three reactants, i.e. the primary aromatic amine hydrochlorides the dicyandiamide, and the adamantane-1-carboxaldehyde, or by refluxing for several hours using ethanol as solvent.

6. Pharmaceutical products which contain compounds of the claims 1, 2 and 3, in any combination with any pharmaceutically accepted excipients.

7. Use of the compounds described in claims 1, 2 and 3, for the preparation of compositions with the purpose of inhibiting the enzyme dihydrofolate reductase or for expression of a gene of a cell with the purpose of inhibiting the proliferation of unwanted cells, belonging to pathogenic microorganisms, human tissues, pathological cells, e.g. tumor cells, like the human bronchopulmonary microcellular tumor cells, or cells belonging to human lens epithelial, primary or secondary cataract, and any pathological cell proliferation.

8. Use of the compounds described in claims 1, 2 and 3, for the preparation of media administered in combination with other pharmaceutical substances, to potentiate the activity of other antimicrobial agents, or the use of non toxic doses (e.g. in combination with antimicrobials, for the protection from the infection or the development of the acquired immunodeficiency syndrome (AIDS).
